(19) United States
(12) Patent Application Publication
(10) Pub. No.: US 2018/0092953 A1
(43) Pub. Date: Apr. 5, 2018

(54) MEDICAL CANNABIS RECOMMENDATION

(71) Applicant: Ronald Douglas Brazil, San Juan Capistrano, CA (US)

(72) Inventor: Ronald Douglas Brazil, San Juan Capistrano, CA (US)

(21) Appl. No.: 15/283,386

(22) Filed: Oct. 1, 2016

(51) Int. Cl. A61K 36/185; A61K 31/352

(52) U.S. Cl. CPC A61K 36/185; A61K 31/352

(57) ABSTRACT

A medical cannabis recommendation method including the following steps: A) A patient meeting with a medical professional; B) evaluation of the patient by the medical professional; C) the medical professional communicates with an individual trained in matching at least one of I) medical symptoms, II) psychological symptoms, and III) physiological symptoms of the patient with a cannabis extract that is best suited for treating at least one of I) medical symptoms, II) psychological symptoms, and III) physiological symptoms; D) the trained individual recommending a cannabis extract for treating the at least one of I) medical symptoms, II) psychological symptoms, and III) physiological symptoms; and E) the medical professional at least one of providing the recommendation and providing the cannabis extract to the patient. The method is preferably designed to help a patient get the beneficial effects that can be provided by the active ingredients in cannabis plants in a way that is targeted to their best benefit by the expertise of a medical professional and at least one of software and a professional trained in matching cannabis extracts to create the best possible benefit for a patient. The method may, however, be used for any suitable purpose.

### BRIEF DESCRIPTION OF THE FIGURES

FIG. 1 is a schematic representation of the system of the first preferred embodiments.

FIG. 2 is a flow chart representation of the system of the first preferred embodiments.

FIG. 3 is a schematic representation of the system of the first preferred embodiments, where the medical professional uses software that includes an algorithm to recommend a cannabis extract that has properties beneficial to the patient.

FIG. 4 is a flow chart representation of the system of the first preferred embodiments, where the cannabis buds are soaked in ethyl alcohol for at least five hours and the alcohol is evaporated off of the extract is evaporated for at least twelve hours, where in one variation at least two extracts are combined, and where the extracts may be heated for decarboxylation, or not heated.

### DESCRIPTION OF THE PREFERRED EMBODIMENTS

The description that follows of the invention's preferred embodiments is meant to enable someone skilled in the prior art to make and use the invention, but is not meant to limit the invention to these preferred embodiments.

### 1. First Preferred Embodiment

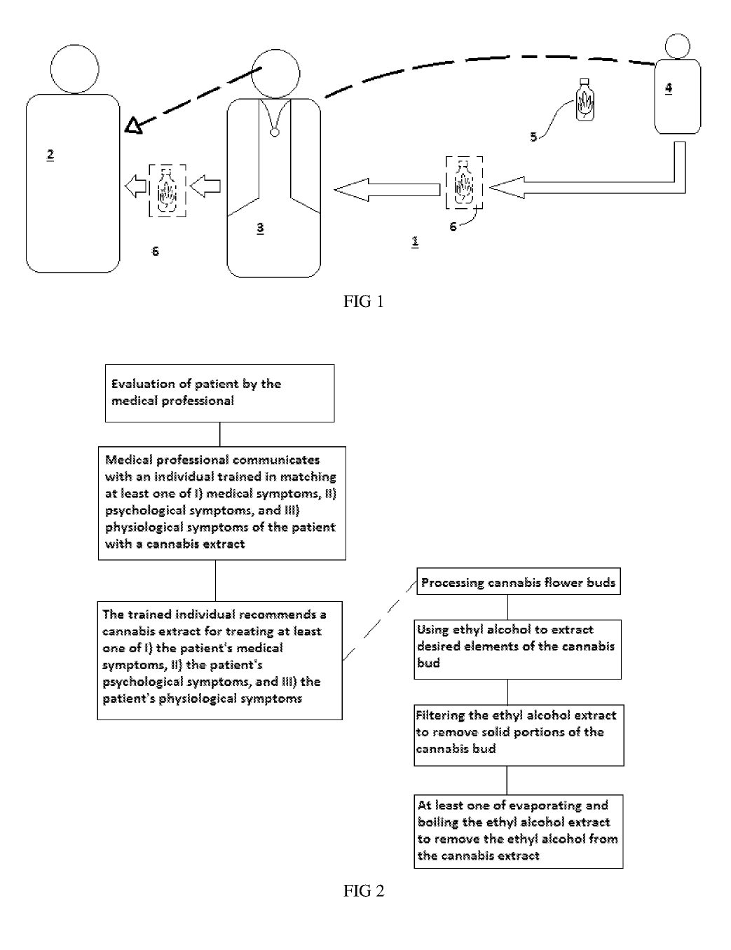

As shown in FIG. 1, the system of the preferred embodiments is a medical cannabis recommendation method 1 including the following steps: A) A patient 2 meeting with a medical professional 3; B) evaluation of the patient 2 by the medical professional 3; C) the medical professional 3 communicates with an individual 4 trained in matching at least one of I) medical symptoms, II) psychological symptoms, and III) physiological symptoms of the patient 2 with a cannabis extract 5 that is best suited for treating at least one of I) medical symptoms, II) psychological symptoms, and III) physiological symptoms; D) the trained individual 4 recommending a cannabis extract 5 for treating the at least one of I) medical symptoms, II) psychological symptoms, and III) physiological symptoms; and E) the medical professional 3 at least one of providing the recommendation 6 and providing the cannabis extract 5 to the patient 2. The method 1 is preferably designed to help a patient 2 get the beneficial effects that can be provided by the active ingredients in cannabis plants in a way that is targeted to their best benefit by the expertise of a medical professional 3 and at least one of software 7 and a professional trained in matching cannabis extracts 5 to create the best possible benefit for a patient 2. The method 1 may, however, be used for any suitable purpose.

As shown in FIGS. 1 and 2, at least one of a patient 2 and a user 2 consults a medical professional 3 at least one of in person, and remotely. The medical professional 3 then conducts at least one of tests, measurements and an interview to determine at least one of I) medical symptoms, II) psychological symptoms, and III) physiological symptoms for the at least one of a patient 2 and a user 2. In a preferred variation, the medical professional 3 then communicates at least one of I) medical symptoms, II) psychological symptoms, and III) physiological symptoms for the at least one of a patient 2 and a user 2 to an individual 4 trained in matching at least one cannabis extract 5 to at least one of I) medical symptoms, II) psychological symptoms, and III) physiological symptoms. The individual 4 preferably is trained in creating benefits to at least one of a user 2 and a patient 2 using the beneficial effects of the active ingredients of a cannabis extract 5. Preferably the user 2 can be at least one of physically, physiologically, medically, and mentally benefited by the recommended cannabis extract 5. The outcome of the cannabis recommendation 6 may, however, be any suitable outcome.

As shown in FIG. 2, the at least one cannabis extract 5 is preferably made by processing cannabis flower buds. In a preferred variation, the cannabis buds are at least one of ground, processed, and powdered. The cannabis buds may, however, be processed in any suitable manner. Preferably ethyl alcohol is then used to extract active ingredients from the cannabis buds. In one preferred variation, the processed cannabis buds are soaked in ethyl alcohol for at least 5 hours. In a preferred variation, the cannabis buds are soaked in ethyl alcohol between 8 and 12 hours. Preferably the ethyl alcohol is then filtered after it is used to extract active ingredients. Preferably the ethyl alcohol mixture is then at least one of boiled and evaporated to remove the ethyl alcohol from the mixture, leaving behind the active ingredients. Preferably this results in an extract that is at least one of an oil, a paste, and a more concentrated tincture. In a preferred variation, the ethyl alcohol mixture is evaporated for at least twelve hours to allow the ethyl alcohol to evaporate out of the mixture. In a further preferred variation, the mixture is allowed to evaporate for at least twenty-four hours. In an alternate variation, the mixture is heated gently to promote faster evaporation of the ethyl alcohol, where the temperature needs to be controlled due to the effects of heating on the active ingredients found in cannabis. In another alternate variation, a reduced pressure environment may be used to accelerate the evaporation of ethyl alcohol from the mixture. In another preferred variation, the ethyl alcohol may be distilled off of the extract mixture, then after distillation the ethyl alcohol mixture may be allowed to further evaporate off the ethyl alcohol. The method 1 may, however, use any suitable method 1 for removing the ethyl alcohol from the ethyl alcohol extract mixture.

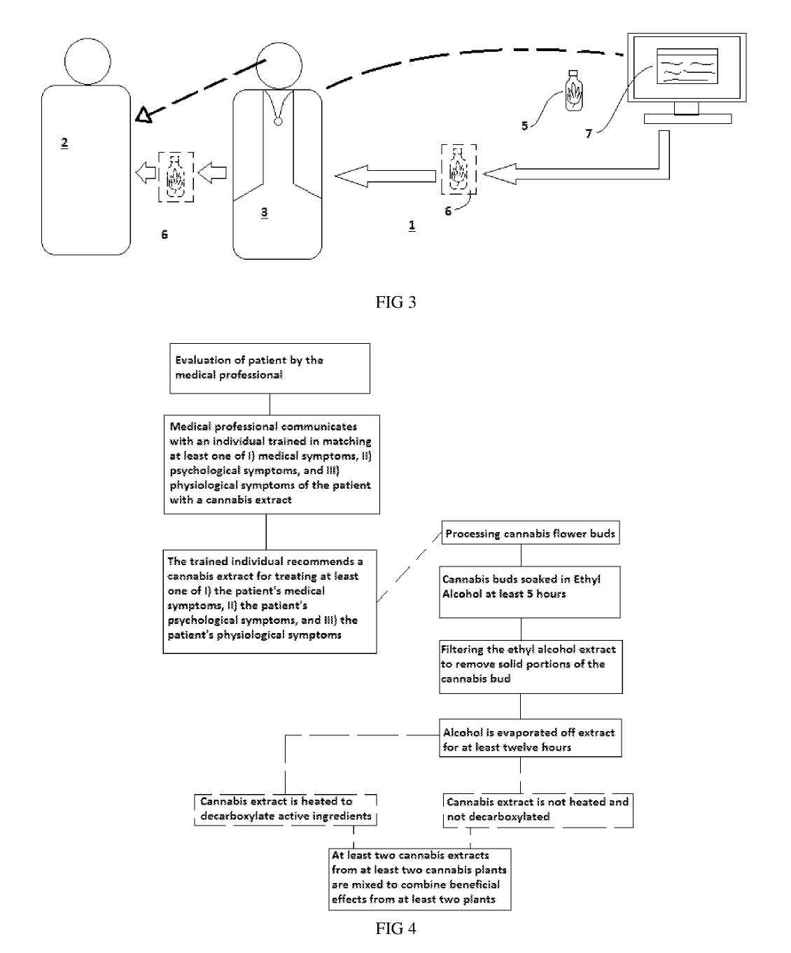

As shown in FIG. 3, in an alternate variation the medical professional 3 inputs at least one of I) medical symptoms, II) psychological symptoms, and III) physiological symptoms related to at least one of a user 2 and a patient 2 into a software 7 program, where the software 7 program includes an algorithm designed to help match at least one of I) medical symptoms, II) psychological symptoms, and III) physiological symptoms to a cannabis extract 5 based on the at least one of I) medical symptoms, II) psychological symptoms, and III) physiological symptoms. Preferably in this variation the software 7 algorithm uses a decision tree to help determine the appropriate at least one cannabis extract 5 to benefit the at least one of a user 2 and a patient 2. The software 7 algorithm may, however, use any suitable method 1 for providing a recommendation 6 for at least one cannabis extract 5 based on at least one of I) medical symptoms, II) psychological symptoms, and III) physiological symptoms. Alternate variations may at least one of 1) look up cannabis extract 5 matches in a database, 2) use statistical methods to correlate cannabis extract 5 matches, and 3) use fuzzy logic algorithms to choose cannabis extract 5 matches.

As shown in FIG. 4, in an alternate preferred variation, at least two cannabis extracts 5 are at least one of blended and used together by at least one of a user 2 and a patient 2. In this preferred variation, the active ingredients of at least two extracts are considered to be beneficial to the at least one of a user 2 and a patient 2, and are recommended by at least one of the trained individual 4 and the recommendation software 7. Preferably this allows better benefiting at least one of I) medical symptoms, II) psychological symptoms, and III) physiological symptoms.

As shown in FIG. 4, in one preferred variation, the cannabis extract 5 and the cannabis buds that are used to create it are not heated at any point, to avoid the decarboxylation of the active ingredients in the resulting extract. The beneficial effects of a decarboxylated cannabis extract 5 often vary from those of a non-decarboxylated cannabis extract 5, so in some circumstances it may be preferable to not heat the cannabis buds and cannabis extract 5 to avoid decarboxylation of the active ingredients. In another preferred variation, at least one of the cannabis extract 5 and the cannabis buds are heated to decarboxylate the active ingredients in the cannabis extract 5. In another preferred variation, at least two cannabis extracts 5 are at least one of blended and recommended for consumption by the at least one of a user 2 and a patient 2, where at least one of the at least two cannabis extracts 5 is non-decarboxylated, and at least one additional of the at least two cannabis extracts 5 is decarboxylated, where this provides the benefits of both decarboxylated and non-decarboxylated cannabis extracts 5. There may, however, be any suitable combination or use of heating, decarboxylation, and non-decarboxylated cannabis extracts 5.

In a preferred variation, a cannabis extract 5 may contain active ingredients included but not limited to THCa, CBDa, THC Delta 9, and CBD. In a preferred variation, the cannabis extract 5 mixture comprises at least 0.1% by weight of at least two of: THCa, CBDa, THC Delta 9, and CBD. Preferably the cannabis extract 5 includes a large enough quantity of at least one of these active ingredients to provide at least one of a physically significant, medically significant, and mentally significant effect on at least one of a user 2 and a patient 2.

As a person skilled in the art will recognize from the previous detailed description and from the figures and claims, modifications and changes can be made to the preferred embodiments of the invention without departing from the scope of this invention defined in the following claims.

1. A medical cannabis prescription production process, comprising:
A) evaluation of a patient by a medical professional;
B) the medical professional matching an identified disease or condition of the patient with a specific cannabis extract produced specifically to treat the identified condition;
B) producing the matched cannabis extract to treat the identified disease or condition by;
i) processing cannabis flower buds using ethyl alcohol to extract desired chemical elements of the cannabis bud;
ii) filtering the ethyl alcohol extract to remove solid portions of the cannabis bud;
iii) evaporating the ethyl alcohol extract to remove the ethyl alcohol from the cannabis extract and
C) and providing the cannabis extract to the patient.

2)-4) (canceled)

5. The medical cannabis prescription process of claim 1, wherein the cannabis buds are soaked in ethyl alcohol for at least 2 hours, wherein the solvent is distilled off of the extract, wherein oil remaining after distilling off the solvent is further allowed to evaporate off solvents for at least one hour.

6) (canceled)

7. The medical cannabis prescription process of claim 1, wherein at least two cannabis extracts from at least two strains of cannabis plant are blended to combine beneficial effects from different strains of cannabis to better treat the identified didease or condition.

8)-12) (canceled)

13. The medical cannabis prescription method process of claim 1, wherein the cannabis extract is heated to decarboxylate the active ingredients.

14)-16) (canceled)

17. The medical cannabis prescription process of claim 7, wherein a first cannabis extract is heated to decarboxylate the active ingredients in the extract, and a second cannabis extract in the mixture is not heated to prevent decarboxylation of the active ingredients in the extract, wherein mixing the first and second provides benefits from both decarboxylated active cannabis ingredients and non-decarboxylated active cannabis ingredients.

18) (canceled)

19. The medical cannabis prescription process of claim 17, wherein the cannabis extract mixture comprises at least 0.1% by weight of at least two of: THCa, CBDa, THC Delta 9, and CBD Delta 9.
